(19) United States
(12) Patent Application Publication
(10) Pub. No.: US 2025/0286405 A1
(43) Pub. Date: Sep. 11, 2025

(54) APPARATUS FOR CHARGING WRIST-MOUNTED MICROPROCESSOR-CONTROLLED ELECTRONIC DEVICES

(71) Applicant: Taction Technology, Inc., Los Gatos, CA (US)

(72) Inventor: John Douglas Steinberg, Millbrae, CA (US)

(21) Appl. No.: 18/987,342

(22) Filed: Dec. 19, 2024

Related U.S. Application Data
(63) Continuation of application No. 18/896,149, filed on Sep. 25, 2024, now abandoned.
Application No. 18/896,149 is a continuation of application No. 18/740,092, filed on Jun. 11, 2024, now abandoned.
Application No. 18/740,092 is a continuation of application No. 18/590,162, filed on Feb. 28, 2024, now abandoned.
Application No. 18/590,162 is a continuation of application No. 18/388,648, filed on Nov. 10, 2023, now abandoned.
Application No. 18/388,648 is a continuation of application No. 18/215,080, filed on Jun. 27, 2023, now abandoned.
Application No. 18/215,080 is a continuation of application No. 18/117,031, filed on Mar. 3, 2023, now abandoned.
Application No. 18/117,031 is a continuation of application No. 17/989,158, filed on Nov. 17, 2022, now abandoned.
Application No. 17/989,158 is a continuation of application No. 17/881,907, filed on Aug. 5, 2022, now abandoned.
Application No. 17/881,907 is a continuation of application No. 17/680,073, filed on Feb. 24, 2022, now abandoned.
(60) Provisional application No. 63/154,771, filed on Feb. 28, 2021.

(51) Int. Cl. H02J 50/10; A61B 5/00; H02J 50/00

(52) U.S. Cl. CPC H02J 50/10; A61B 5/681; H02J 50/005

(57) ABSTRACT

An apparatus for use with a wrist-mounted device comprising biometric features such as pulse rate, blood pressure or other biometric measurements, such as a smart watch, wherein the apparatus permits the battery in the wrist-mounted device to be charged while the device is on the user's wrist, thereby permitting the wrist-mounted device to continue to record biometric signals while the wrist-mounted device is being charged.

### INCORPORATION BY REFERENCE TO ANY PRIORITY APPLICATIONS

Any and all applications, if any, for which a foreign or domestic priority claim is identified in the Application Data Sheet of the present application are hereby incorporated by reference under 37 CFR 1.57.

### BACKGROUND OF THE INVENTION

Until the 1990s, very few people had personal, battery-powered electronic devices that were controlled by even moderately sophisticated microprocessors. The first mobile phones were just phones, though the ability send simple text messages soon followed. Adequate run time was not generally a major issue. Early notebook computers had the ability perform complex tasks, but run time when disconnected from a wall was often a problem. But with the exception of people working on long airplane flights, that was generally a tolerable issue-people did not use those devices 24/7, and could usually plug them in as needed.

As “dumb” phones became smart phones, with a myriad of applications, battery life became a serious issue. People carry their smart phones all day long, and now even bring their phones into the bedroom at night. Because heavy use can consume the entire charge in a smart phone's internal battery in just a few hours, many vendors have offered smart phone cases that include secondary batteries that can be used to power the phone and/or recharge its internal battery. But boarding areas at airports still have many people seated on the floor to be near electrical outlets in order to charge their smart phones.

The next stage in the path to ubiquitous, always-on personal devices has been smart watches. Remarkable progress in miniaturization of processors, memory, displays, etc. allows the latest watches to perform many of the tasks that once required a desktop computer. But that progress in miniaturization has not been matched by progress in battery power density. Smart watches often struggle to make it through 24 hours on a single charge. This contrasts greatly with traditional quartz watches, which can easily run for an entire year or more on a single tiny battery.

And unlike PCs or smart phones, which can often be used while plugged into a charger, it has been impractical to simultaneously wear and charge a smart watch. That makes it difficult to consistently use some of the most useful features of smart watches, which can monitor critical health indicators such as heart rate, ECG, blood pressure, sleep patterns, etc. 24/7, even while sleeping.

Larger batteries could at least partially address the limitations of the small internal battery. But smart watches already tend to be bulkier and heavier than the traditional watches they are replacing. Many consumers would reject larger, heavier wrist-mounted electronic devices.

For a variety of reasons, many smart watches use inductive charging. This is accomplished by lining up a coiled wire in the smart watch with a matching coil in a charging device. When alternating current is passed through the coil in the charging device, it induces an alternating current in the device to be charged. This alternating current is generally rectified back to direct current so that it can be used to power the device, or be used to charge a battery in the second device. Though inductive charging is often less efficient than direct charging, it has the advantage that there is no direct physical electrical connection between the two devices. This can simplify and/or improve the waterproofing of the devices. Magnets may be used to help align the two coils securely. These inductive chargers are generally either plugged directly into an electrical outlet, or into another device such as a PC.

These chargers are effective at delivering power sufficient to recharge the smart watches. Some such chargers are designed to allow a user to see the watch face while it is charging. But it is currently impossible or at least impractical to use those chargers and wear the watch simultaneously. And because most watches must be charged almost every night to get them though a full day's use, this means that users cannot track sleep patterns, heart rate, etc. while the device is on the nightstand charging.

Thus there is a need for a cordless, battery powered smart watch charger that permits charging of the smart watch while the watch remains on the user's wrist, without obscuring biometric sensors.

### SUMMARY

A recharging module comprising at least a battery and an inductive charging coil is configured to permit its coil to be placed between a user's wrist and the smart watch. Magnets and/or mechanical means maintain alignment between the charging coil in the module and the smart watch, enabling the module to charge the smart phone. When the watch has been recharged, the module can in turn be removed from the user's wrist and recharged by either being plugged into a power source, or by being placed on or in a docking station that may be placed on a desk or nightstand and left attached to a power source.

In one embodiment, the subject invention comprises an apparatus for charging a microprocessor-controlled wrist-mounted electronic device, wherein said wrist mounted-device comprises at least an optical sensor that measures at least a biological function, and further comprises at least a rechargeable battery and at least a first coil of conductive wire configured to permit said wrist-mounted electronic device to be inductively charged when said first coil of conductive wire in said wrist-mounted electronic device is placed in operative proximity to an energized coil in a charging apparatus, said apparatus comprising: a first module comprising at least a second coil of conductive wire configured so as to permit said second coil of conductive wire to be aligned in operative proximity with said at least said first coil of conductive wire in said wrist-mounted electronic device; at least a rechargeable battery; an annular ring sized to locate said at least a second coil of conductive wire in operative proximity with said first at least a coil of conductive wire; an opening within said annular ring that permits at least said optical sensor to measure at least said biological function; a second module comprising: at least a third coil of conductive wire configured so as to be aligned in operative proximity with said at least said second coil of conductive wire in said first module when said second coil of conductive wire in said first module is not in operative proximity with said first coil of conductive wire in said wrist-mounted electronic device.

In another embodiment, the subject invention comprises an apparatus for charging a microprocessor-controlled wrist-mounted electronic device, wherein said wrist mounted-device comprises at least an optical sensor that measures at least a biological function, and further comprises at least a rechargeable battery and at least a first coil of conductive wire configured to permit said wrist-mounted electronic device to be inductively charged when said first coil of conductive wire in said wrist-mounted electronic device is placed in operative proximity to an energized coil in a charging apparatus, said apparatus comprising a first module comprising at least a second coil of conductive wire configured so as to permit said second coil of conductive wire to be aligned in operative proximity with said at least said first coil of conductive wire in said wrist-mounted electronic device; at least a rechargeable battery; an annular ring sized to locate said at least a second coil of conductive wire in operative proximity with said first at least a coil of conductive wire; an opening within said annular ring that permits at least said optical sensor to measure at least said biological function; a first plurality of exposed electrical contacts through which electrical current may be passed to said at least a rechargeable battery; a second module comprising: a second plurality of exposed electrical contacts through which electrical current may be passed; an internal power supply, wherein said first plurality of exposed electrical contacts can be connected to said second plurality of electrical contacts when said first module and said second module are placed in physical contact.

In another embodiment, the subject invention comprises an apparatus for charging a microprocessor-controlled wrist-mounted electronic device, wherein said wrist mounted-device comprises at least an optical sensor that measures at least a biological function, and further comprises at least a rechargeable battery and a first plurality of external electrically conductive contacts configured to permit said wrist-mounted electronic device to be charged when said external conductive contacts are placed in operative physical contact with a charging apparatus, said apparatus comprising a first module comprising: at least a second plurality of external electrically conductive contacts configured so as to permit said second plurality of electrically conductive contacts to be aligned in operative proximity with said at first plurality of external electrically conductive contacts in said wrist-mounted electronic device; at least a rechargeable battery; an annular ring sized to locate said wrist-mounted electronic device so that said first plurality of conductive contacts make contact with said second electrically conductive contacts; an opening within said annular ring that permits at least said optical sensor to measure at least said biological function; a second module comprising: at least a third plurality of electrically conductive contacts configured so as to be aligned in contact with at least a plurality of electrically conductive contacts on said first module when said second module is in operative proximity with said first module is mated with said second module.

In another embodiment, the subject invention comprises an apparatus for charging a microprocessor-controlled wrist-mounted electronic device, wherein said wrist mounted-device comprises at least an optical sensor that measures at least a biological function, and further comprises at least a rechargeable battery and at least a multi-layer self-resonant structure comprising multiple conductive layers separated by dielectric layers configured to permit said wrist-mounted electronic device to be charged when said first multi-layer self-resonant structure comprising multiple conductive layers separated by dielectric layers in said wrist-mounted electronic device is placed in operative proximity to an energized coil in a charging apparatus, said apparatus comprising a first module comprising at least a second multi-layer self-resonant structure comprising multiple conductive layers separated by dielectric layers configured so as to permit said second multi-layer self-resonant structure comprising multiple conductive layers separated by dielectric layers to be aligned in operative proximity with said at least said first multi-layer self-resonant structure comprising multiple conductive layers separated by dielectric layers in said wrist-mounted electronic device; at least a rechargeable battery; an annular ring sized to locate said at least a second multi-layer self-resonant structure comprising multiple conductive layers separated by dielectric layers in operative proximity with said first multi-layer self-resonant structure comprising multiple conductive layers separated by dielectric layers; an opening within said annular ring that permits at least said optical sensor to measure at least said biological function; a first plurality of exposed electrical contacts through which electrical current may be passed to said at least a rechargeable battery; a second module comprising: a second plurality of exposed electrical contacts through which electrical current may be passed; an internal power supply, wherein said first plurality of exposed electrical contacts can be connected to said second plurality of electrical contacts when said first module and said second module are placed in physical contact.

In another embodiment the invention comprises a process for intermittently charging a smart watch with an apparatus comprising at least a battery, a microprocessor and a coil that is capable of passing a charge to a coil in the smart watch while worn by a user, such that the microprocessor alternately charges the smart watch and pauses charging to permit biometric features in said smart watch to function.

### BRIEF DESCRIPTION OF THE DRAWINGS

FIG. 1a shows a perspective view and 1b shows a plan view of the back face of a wrist-worn electronic device such as a smart watch as is found in prior art.

FIG. 2 illustrates external aspects of an aspect of an embodiment of the invention.

FIG. 3a shows a smart watch and an embodiment of the subject invention as they would be aligned when connected and held on a user's arm.

FIG. 3b illustrates a smart watch and an embodiment of the subject invention as worn on a user's wrist/forearm.

FIG. 4 is a simplified view of some of the key internal components of an exemplary embodiment of the subject invention.

FIG. 5a illustrates an embodiment of a wearable charging module mounted on a base unit.

FIG. 5b illustrates an embodiment of a wearable charging module that may be configured to be chargeable without a base unit.

FIGS. 6a through 6e illustrate additional mechanical means for retaining a smart watch in the proper orientation relative to exemplary embodiments of the subject invention.

FIG. 7 illustrates another mechanical means that can be used to maintain alignment between a smart watch and an exemplary embodiment of the subject invention.

FIG. 8 illustrates an alternate embodiment in which one or more coils for charging a smart watch may be located in a wristband of the smart watch.

### DETAILED DESCRIPTION

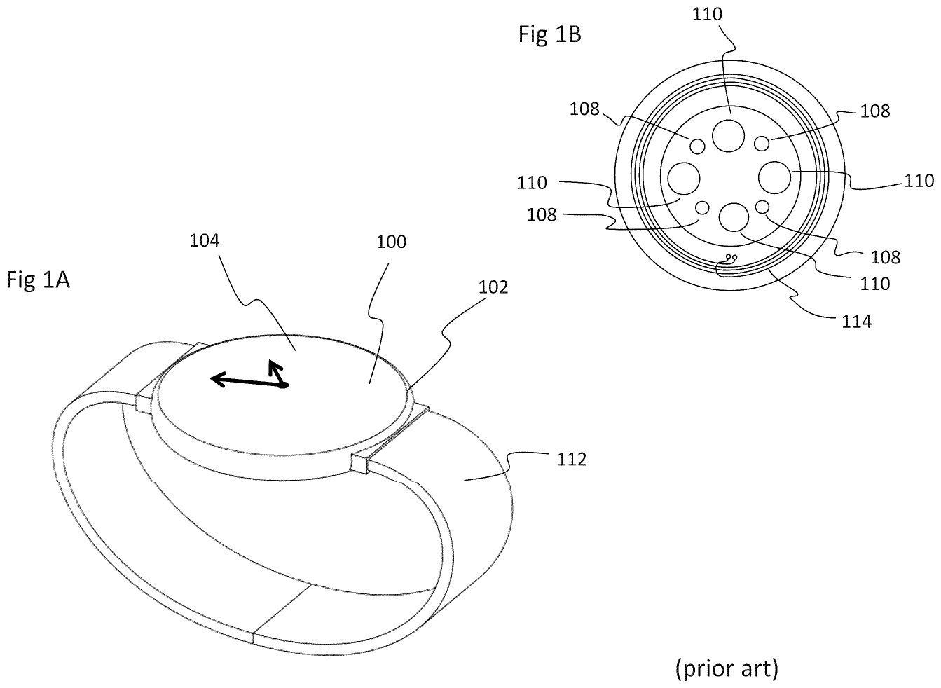

FIG. 1a shows a perspective view and 1b shows a plan view of the back face of a wrist-worn electronic device such as a smart watch 100 as is found in prior art. Watch chassis 102 includes a display 104, which may be used to show a digital simulation of a conventional watch, as shown, displays of a variety of other information, such as weather, heart rate, text alerts, other information, or various combinations thereof. In addition, watch 100 may include biometric features on the back or inside face of the watch that normally contacts a user's wrist. These may include, for example, light sources 108 such as red, green and infrared light sources, and optical sensors 110 for measuring biological functions such as pulse rate, blood pressure, and/or blood oxygen. Smart watch 100 may also include non-optical sensors for measuring additional biological functions such as body temperature, blood pressure or other parameters. Wristband 112 holds smart watch 100 on the user's wrist. Wristband 112 may also contain additional sensors.

Smart watch 100 includes one or more internal microprocessors. Many such smart watches also include one or more wire coils 114 that permit inductive charging of the smart watch. Smart watch 100 also includes at least an internal battery. It likely also includes circuitry that can convert alternating current provided through inductive charging to direct current that can be applied to store power in the onboard battery. Other smart watches use a physical connector to attach a charging cable. These devices likely do not require circuitry that performs the rectification from alternating to direct current. And other smart watches may include exposed physical contacts that can be connected with matching contacts in a charging device.

Smart watch 100 as shown in FIGS. 1a and 1b is round when viewed from above display 104. However, smart watch 100 may take different form factors, such as square, rectangular, ovoid, or other shapes.

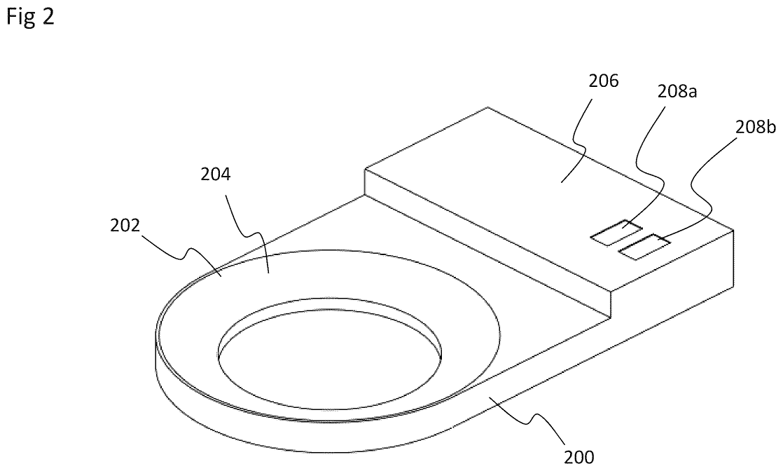

FIG. 2 illustrates external aspects of an aspect of an embodiment of the invention. Module 200 includes a ring 202, which holds an internal wire coil used for inductive charging. Annular ring 202 may include means to help locate the wire coil in the correct position relative to the wire coil in the smart watch it is to be paired with. Such means may include one or more of (a) a conical or spherical segment face 204 to help center smart watch 100 on the appropriate portion of module 200, (b) a magnet or magnets (not shown) to help pull the smart watch into alignment by aligning itself with a magnet or magnetically permeable material in a matching location in the smart watch, and (c) additional mechanical alignment features, such as ridges or arms (not shown in this figure). Ring 202 is open at its center to permit biometric features 108 and 110 in smart watch 100 to operate even while the smart watch 100 is being charged with module 200. Alternatively, at least a portion of the center section may be comprised of a transparent material, such as glass or plastic. While this may not allow all possible biometric sensors to operate accurately (such as temperature sensors), it will permit some forms of sensors to do so. A transparent center section will also simplify mounting one or more locating magnets at the center of ring 202 for use with smart watches that include a locating magnet in the center of the back of the device.

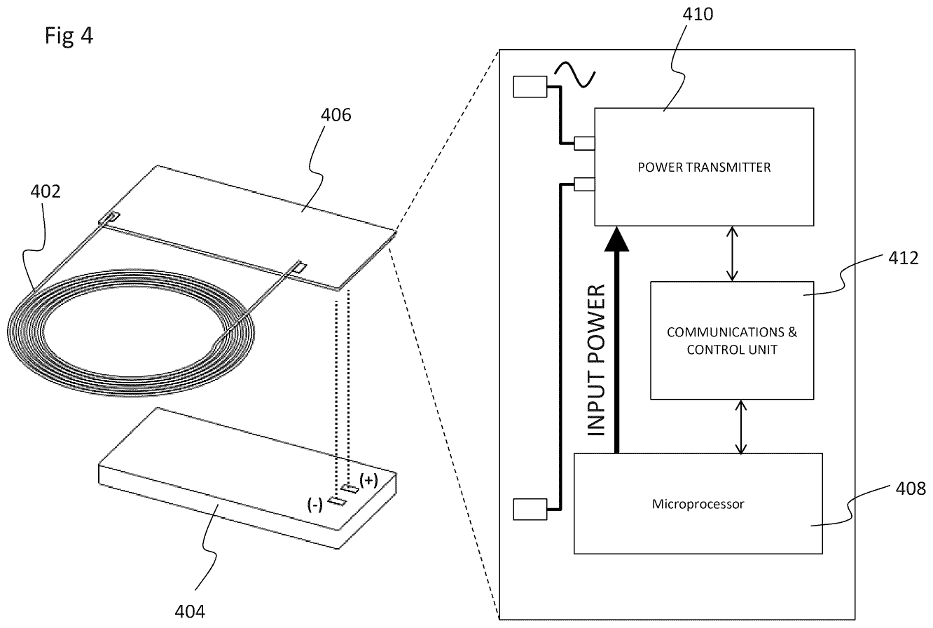

In this embodiment, module 200 also includes battery compartment 206. Battery compartment preferably includes one or more rechargeable batteries, such as lithium ion or nickel metal hydride, as shown in FIG. 4. Module 200 may also include one or more status lights 208a and 208b to indicate to a user the status of parameters such the state of charge of the batteries in module 200, whether module 200 is currently charging smart watch 100, etc. Such lights may be LEDs, OLEDs or other suitable light sources. Alternatively, module 200 may include a monochrome or color display or touch-sensitive display that can provide a richer interface to monitor and/or control its functions.

In at least an alternative embodiment, module 200 may include exposed physical contacts that align with and touch exposed physical contacts in smart watch 100 when module 200 and smart watch 100 are mated.

In at least an alternative embodiment, module 200 may include an electrical connector that mates with a physical connector in smart watch 100. Examples of such physical connectors include various USB (Universal Serial Bus) versions such as micro-USB, USB-C, Apple's Lighting, etc.

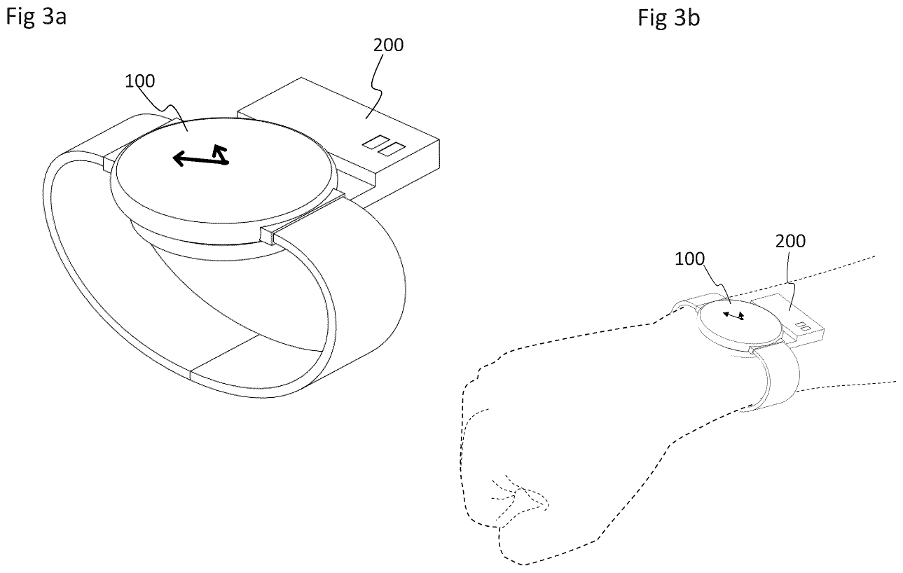

FIG. 3a shows smart watch 100 and module 200 as they would be aligned when connected and held on a user's arm. The back/inner face of smart watch 100 may have a conic or spherical section or spherical cap-shaped “male” region that can be mated with a similarly shaped “female” section 204 in module 200. Loading of that interface may be provided by strap 112, which in effect may push the back/inner face of smart watch 100 into section 204 of module 200, and by module 200 itself being pushed outward toward smart watch 100 by being wedged between the user's arm and smart watch 100.

FIG. 3b illustrates smart watch 100 and module 200 as worn on a user's wrist/forearm.

FIG. 4 is a simplified view of some of the key internal components of an exemplary embodiment of module 200. Wire coil 402 is located within ring 202. The ends of the coil are connected to at least PC board 406 capable of at least converting the direct current from the positive and negative terminals of battery 304 into the alternating current required to transmit energy through wire coil 302. PC board 406 may include at least a microprocessor 408, which can be used to manage the state of charge of internal battery 404, optimize charging of smart watch 100, etc. PC board 406 also includes one or more components comprising a power transmission module 410. Power transmission module 410 may comprise an oscillator to generate a waveform of the appropriate frequency and an amplifier (which may itself comprise a simple transistor) to deliver the waveform to coil 402 with sufficient current to accomplish charging of the smart watch 100. PC board 406 may also include a communications and control module 412 to assist in managing the operation of power transmitter 410. Communications and control module 412 may include means for sensing current flowing through coil 402, and to sense the additional inductance of smart watch 100 when it is in operative proximity to coil 402. Battery 304 is preferably sized so that it is large enough to comfortably charge the internal battery in smart watch 100 despite the inherent losses in inductive charging, which may be as high as 50% or more. Because it is likely that users will use module 200 to charge smart watch 100 while sleeping, it is important that module 200 be capable, at a minimum, of charging smart watch 100 in a single session, and that such a charge cycle be completed within a few hours.

If coil 402 is also used to charge battery 404, additional power receiver components may be included such as a power pick-up unit that includes AC to DC power rectification and low-pass filtering. A communications and control unit may also be included that may communicates via the coil 402 with a power-transmitting base unit. The communications and control unit may also sense and manage battery charge, run indicator lights, and so on.

Microprocessor 408 may also be used to control the display or indicator lights 208a and 208b, in module 200, and, in some embodiments, WiFi and/or Bluetooth communication means. Module 200 may also include electrical contact points to allow direct charging while module 200 is placed in its base unit for charging. It may also include circuitry to permit internal battery 304 to be charged by an external inductive charger, and circuitry that can automatically switch the function of the internal battery 404 and wire coil 402 from being charged by another power source to charging smart watch 100.

In an alternate embodiment, if the module is to be paired with a smart watch 100 that uses a plurality of coils for inductive charging, module 200 may also include a plurality of coils.

Wire coil 402 is shown in FIG. 4 as round in shape, which is the optimal shape if the coil in smart watch 100 is also round. In alternate embodiments, wire coil 402 may be oval, square, rectangular, or another geometric configuration.

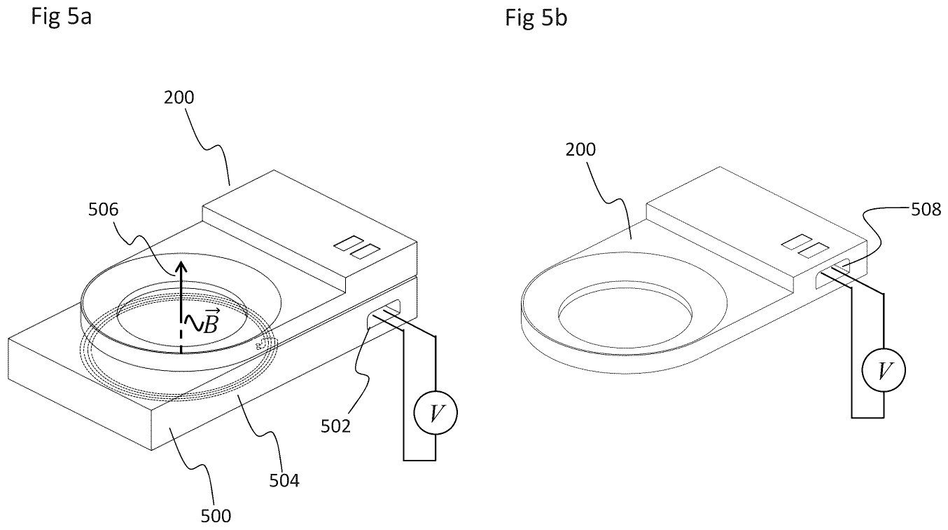

FIG. 5a illustrates module 200 mounted on base unit 500. Base unit 500 includes a means for connecting to a power source, such as USB type A socket 502. Other form factors, including other USB form factors or coaxial connectors may be used. However, base unit 500 may be hardwired with a power cord that may be plugged directly into a wall socket. Base unit may include an inductive charging coil 504 to be used to charge battery 404 through coil 402, as indicated by the time-varying magnetic field B 506 produced by the inductive charging coil. Alternatively, base unit 500 may be configured to charge module 200 directly through electrical contacts in base unit 500 that match with electrical contacts in module 200. In the currently preferred embodiment, module 200 would be water resistant, which is more easily accomplished by both charging module 200 and using model 200 to charge smart watch 100 via inductive charging, because no exposed wires or contacts would be required.

Base unit 500 may also include inductive or other means to charge other devices, such as earphones, smart phones, etc.

Base unit 500 and module 200 may also be configured so that smart watch 100 may be charged by module 200 even when smart watch 100 is not being worn on the user's arm. This may be accomplished by aligning smart watch 100 with module 200 as discussed above while module 200 is mounted on base unit 500. This could be accomplished either by transferring power from base unit 500 to battery 404 in module 200, and then to the battery in smart watch 100, or may be accomplished by bypassing the battery in module 200 and passing power to coil 402 and then into smart watch 100.

Because it is anticipated that one of the ways the subject invention will used is for a person who owns a smart watch to attach module 200 to smart watch 100 while the user is sleeping while wearing the smart watch, it will be important that coil 302 in module 200 remain precisely aligned with the charging coil in smart watch 100 even if a user tosses and turns while sleeping, which could dislodge module 200 from smart watch 100 or just alter alignment enough to hamper charging. Some locating force may be provided by a combination of the shapes of the back face of the smart watch 100 and the shape of ring 202. Additional locating force may be provided by watchband 110, which can help seat smart watch 100 in the ring 202.

However, in applications such as those in which water resistance is less of a concern, module 200 may be configured to be chargeable without a base unit, as shown in FIG. 5b. Module 200 may be fitted with a connector such as a USB socket 508 to permit a conventional charger to be plugged directly into module 200.

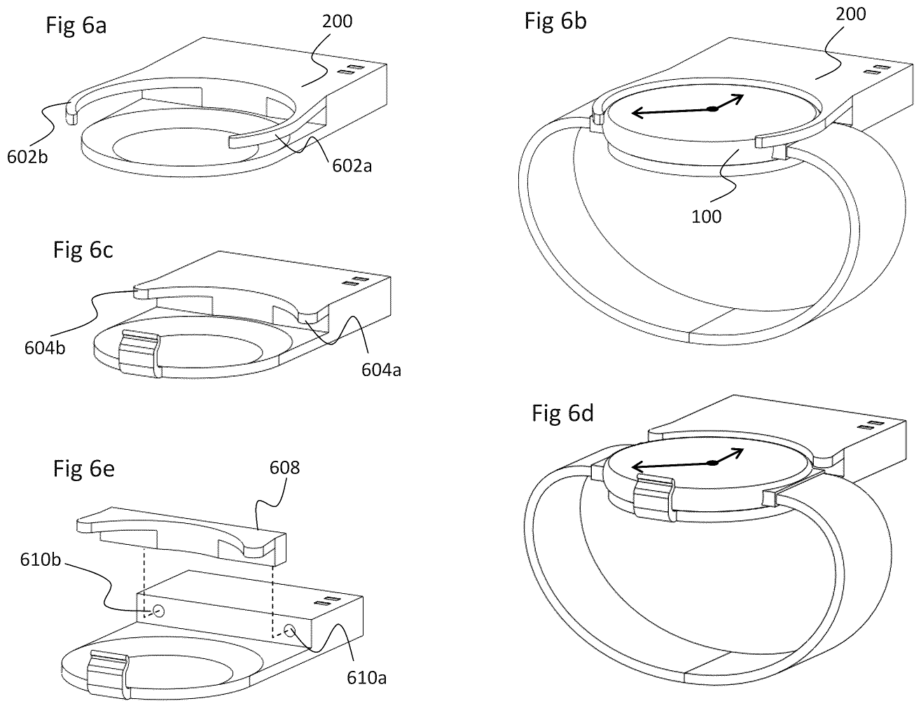

FIG. 6a through 6e illustrate additional mechanical means for retaining smart watch 100 in the proper orientation relative to module 200. In the embodiment shown in FIG. 6a, module 200 includes two arms 602a and 602b that extend outward from battery compartment 206 toward ring 202. The static opening between arms 602a and 602b is preferably slightly smaller than the outside diameter of smart watch 100. (If the body of smart watch 100 is a rounded rectangle or rounded square, arms 602a and 602b will preferably be sized to be slightly smaller than those shapes.) When a user slides ring 202 of module 200 under smart watch 100 while smart watch 100 is on the user's wrist, the body of smart watch 100 pushes arms 602a and 602b outward slightly. When smart watch 100 is fully seated over ring 202, arms 602a and 602b revert to their static unloaded positions, or may be held slightly apart by smart watch 100. The gap between arms 602a and 602b and ring 202 provide a space for wristband 112. FIG. 6b illustrates smart watch 100 as located relative to module 200 in part by arms 602a and 602b.

FIGS. 6c-e illustrate an alternative embodiment of mechanical means for locating smart watch 100 relative to module 200. As shown in FIG. 6c, in this embodiment, protrusions 604a and 604b are shaped to conform to features of the case of smart watch 100 such that smart watch 100 is inhibited from moving relative to module 200 in certain directions. One or more upward protrusions from ring 202 may assist in properly locating ring 202 relative to smart watch 100.

FIG. 6d illustrates smart watch 100 as located on ring 202 of module 200 by protrusions 604a, 604 b, and 604c.

One complication for efficient manufacturing of the subject invention is that smart watches are sold in a variety of sizes and shapes. It may be desirable to partially both make a relatively standardized charging module for a variety of smart watches, and to make the product somewhat “future-proof” by allowing a consumer to easily adapt the device to newer smart watches if the form factor changes. FIG. 6e illustrates an embodiment of module 200 that permits such adaptation. Module 200 includes interchangeable adapter 608, which may be attached to module 200 with tabs or pins (not shown) which may be inserted into slots or recesses 610a and 610b. Module 200 may be sized so that a variety of adapters 608 may be used to achieve a proper fit with different smart watches 100.

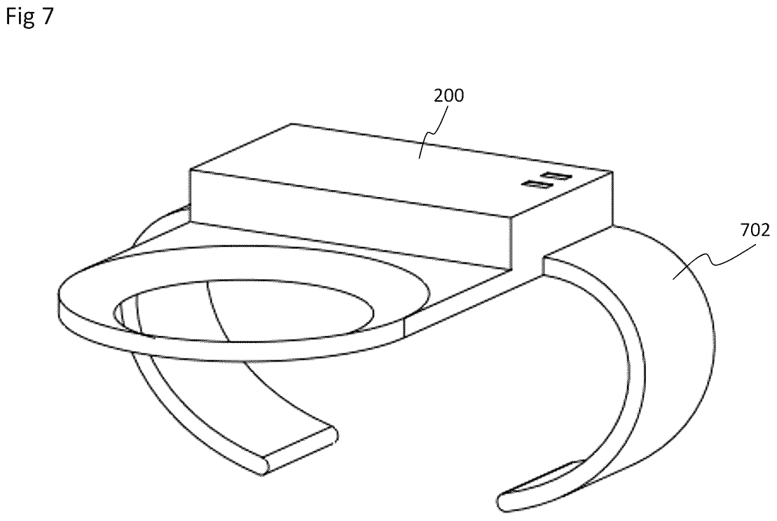

FIG. 7 illustrates another mechanical means that can be used to maintain alignment between smart watch 100 and module 200. Partial or complete wristband 702 may be attached to module 200 to keep module 200 from moving relative to smart watch 100 when the partial or complete wristband is attached to the wrist or forearm of the user wearing smart watch 100. If partial wristbands are provided as shown in FIG. 7, the partial wristbands 702 should be comprised of a resilient material. If a complete wristband is provided it should either be elastic, to allow it to be slipped over the hand and smart watch 100, or provided with a fastener as in a traditional watchband so that it can be fastened after placing module 200 in the proper position.

In an alternative embodiment, instead of placing a relatively bulky battery in a raised compartment 206, one or more flexible or formable batteries could be located in wristband or wristbands 702.

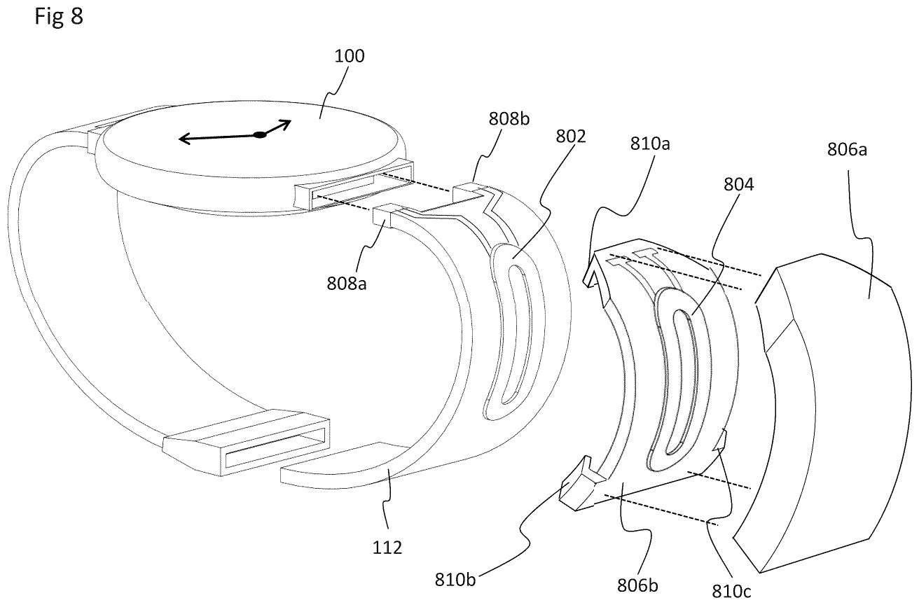

In another alternate embodiment, shown in FIG. 8, one or more coils 802 for charging smart watch 100 may be located in wristband 112. Such a coil or coils could be a loop or loops located entirely within a section of the wristband 110, such that each such coil in effect lies atop the user's wrist, or may wrap around the circumference of the user's wrist, presenting a single large-diameter coil. In both such cases, the proper orientation for the charging coil or coils 804 in a charging module comprising parts 806a and 806b (shown separated to reveal coil 804) would be a coil or coils that wrap around the wristband of smart watch 100 and that are placed directly over said coil or coils when module 800 and smart watch 100 are both worn on the user's wrist. In this embodiment, module 806 would contain a battery and electronics similar to those discussed above relative to other embodiments. Conductive contacts 808a and 808b in wristband 112 connect coil 802 with the battery located in smart watch 100.

One method for removably attaching charging module 806 to wristband 112 is a plurality of hooks to partial wrap around wristband 112 such as hooks 810a, 810b and 810c.

In some embodiments, magnets may be used to help to locate the wire coil 804 in module 806 in proper orientation relative to the wire coil in smart watch 100. In some smart watches, a magnet may be located at the center of the back face of the smart watch. For such cases, a magnet can be placed in the center of an otherwise clear section inside ring 202 of module 200. In other applications, magnets may be placed around the perimeter of the back face of smart watch 100 and just outside wire coil 302 in module 200.

If the portion of the wristband in which coil 802 is located is relatively rigid, wear and tear on the copper wiring that comprises coil 802 will be minimized. If the portion of the wristband where coil 802 is located is flexible, care must be taken to ensure that repeated bending of the wristband does not cause the wiring of coil 802 to work harden and eventually fail.

In an alternate embodiment, smartwatch band 112 may include means for converting the alternating current transmitted to coil 802 into direct current, which may then be conveyed to smart watch 100.

In an alternative embodiment, a charging coil may be attached to or integrated into the smartwatch band such that the charging coil is held between the user's wrist and the charging coil of the smart watch.

In another alternate embodiment, module 200 may provide means for being connected directly to a conventional direct (non-inductive) charger, so that a base unit 400 is not required.

In another alternate embodiment, module 200 may provide means for being inductively charged with a general purpose inductive charger as are commonly available for devices such as smart phones, so that a base unit 400 is not required.

In some smart watches, biometric features may normally be switched off while the smart watch is being charged, since prior art chargers require that the smart watch be removed from the user's wrist to be charged. This can defeat one of the purposes of using the subject invention. Ideally, the software in the smart watch will permit the biometric features to continue to operate while the watch is being charged with the subject invention. However, where that is not the case, it will be possible to operate the subject invention by charging on an intermittent basis. For example, microprocessor 408 may be used to cycle charging of smart watch 100 on a one minute on, one minute off cycle, or some other periodic regime which allows biometric features to continue to collect data on an intermittent basis. While intermittent data collection is less preferable than constant monitoring, it can still allow a user to track parameters like sleep patterns, heartrate and the like with reasonable accuracy.

In an alternate embodiment, the subject invention may comprise a plurality of coils embedded in the wristband of a smartwatch, and a battery-powered charger that fits over those coils.

In an alternate embodiment, instead of using a continuous coiled wire in each of the smartwatch (or watchband) and charging module, each of these components may be coupled to the other for charging using a multi-layer self-resonant structure such as the one described in U.S. Pat. No. 10,707,011. Such a structure may include a multilayer conductor comprising a separation dielectric layer and a plurality of conductor layers stacked in an alternating manner.

What is claimed is:

1. An apparatus for charging a microprocessor-controlled wrist-mounted electronic device, wherein said wrist mounted-device comprises at least an optical sensor that measures at least a biological function, and further comprises at least a rechargeable battery and at least a first coil of conductive wire configured to permit said wrist-mounted electronic device to be inductively charged when said first coil of conductive wire in said wrist-mounted electronic device is placed in operative proximity to an energized coil in a charging apparatus, said apparatus comprising:
a first module comprising:
at least a second coil of conductive wire configured so as to permit said second coil of conductive wire to be aligned in operative proximity with said at least said first coil of conductive wire in said wrist-mounted electronic device;
at least a rechargeable battery;
an annular ring sized to locate said at least a second coil of conductive wire in operative proximity with said first at least a coil of conductive wire;
an opening within said annular ring that permits at least said optical sensor to measure at least said biological function; and
a second module comprising: at least a third coil of conductive wire configured so as to be aligned in operative proximity with said at least said second coil of conductive wire in said first module when said second coil of conductive wire in said first module is not in operative proximity with said first coil of conductive wire in said wrist-mounted electronic device.

2. An apparatus as in claim 1 in which said second coil of conductive wire is round.

3. An apparatus as in claim 1 in which the alignment of said second coil of conductive wire with said first coil of conductive wire is assisted by at least a first magnet in said wrist-mounted electronic device aligning with at least a magnet in said first module mounted in first module so that its polarity is opposite to the polarity of the at least a magnet in said wrist-mounted electronic device.

4. An apparatus as in claim 1 in which the alignment of said second coil of conductive wire with said first coil of conductive wire is assisted by mechanical means of locating said first module relative to said wrist-mounted electronic device, said mechanical means comprising at least an arm that retains at least a portion of said wrist-mounted electronic device, wherein said plurality of arms are sized and positioned such that said plurality of arms produce a compressive force against said wrist-mounted electronic device when said wrist-mounted electronic device and said first module are placed so that said first coil of conductive wire and said second coil of conductive wire are placed in operative proximity.

5. An apparatus as in claim 1 in which the alignment of said second coil of conductive wire with said first coil of conductive wire is assisted by mechanical means of locating said first module relative to said wrist-mounted electronic device, said mechanical means comprising a wristband attaching said first module to the wrist or forearm of a user while also wearing said wrist-mounted electronic device.

6. An apparatus as in claim 1 in which said first module may be electrically connected to said second module with a plurality of exposed electrical contacts on each of said first module and said second module, through which electrical current can flow when said plurality of electrical contacts in said first module are in direct physical contact with said electrical contacts in said second module.

7. An apparatus as in claim 1 in which said battery or batteries in said first module may be electrically charged by placing said second coil of conductive wire in operative proximity with said third coil of conductive wire in said second module through induction.

8. An apparatus as in claim 1 in which each of said first module and said second module contain a single coil of conductive wire used for inductive charging.

9. An apparatus as in claim 1 in which each of said first module and said second module contain a plurality of coils of conductive wire used for inductive charging.

10. An apparatus as in claim 1 in which said second module is capable of charging a plurality of devices.

11. An apparatus for charging a microprocessor-controlled wrist-mounted electronic device, wherein said wrist mounted-device comprises at least an optical sensor that measures at least a biological function, and further comprises at least a rechargeable battery and at least a first coil of conductive wire configured to permit said wrist-mounted electronic device to be inductively charged when said first coil of conductive wire in said wrist-mounted electronic device is placed in operative proximity to an energized coil in a charging apparatus, said apparatus comprising:
a first module comprising:
at least a second coil of conductive wire configured so as to permit said second coil of conductive wire to be aligned in operative proximity with said at least said first coil of conductive wire in said wrist-mounted electronic device;
at least a rechargeable battery;
an annular ring sized to locate said at least a second coil of conductive wire in operative proximity with said first at least a coil of conductive wire;
an opening within said annular ring that permits at least said optical sensor to measure at least said biological function;
a first plurality of exposed electrical contacts through which electrical current may be passed to said at least a rechargeable battery;
a second module comprising: a second plurality of exposed electrical contacts through which electrical current may be passed;
an internal power supply; and
wherein said first plurality of exposed electrical contacts can be connected to said second plurality of electrical contacts when said first module and said second module are placed in physical contact.

12. An apparatus for charging a microprocessor-controlled wrist-mounted electronic device, wherein said wrist mounted-device comprises at least an optical sensor that measures at least a biological function, and further comprises at least a rechargeable battery and a first plurality of external electrically conductive contacts configured to permit said wrist-mounted electronic device to be charged when said external conductive contacts are placed in operative physical contact with a charging apparatus, said apparatus comprising:
a first module comprising:
at least a second plurality of external electrically conductive contacts configured so as to permit said second plurality of electrically conductive contacts to be aligned in operative proximity with said at first plurality of external electrically conductive contacts in said wrist-mounted electronic device;
at least a rechargeable battery;
an annular ring sized to locate said wrist-mounted electronic device so that said first plurality of conductive contacts make contact with said second electrically conductive contacts;
an opening within said annular ring that permits at least said optical sensor to measure at least said biological function; and
a second module comprising: at least a third plurality of electrically conductive contacts configured so as to be aligned in contact with at least a plurality of electrically conductive contacts on said first module when said second module is in operative proximity with said first module is mated with said second module.
